(19) United States
(12) Patent Application Publication
(10) Pub. No.: US 2008/0268506 A1
(43) Pub. Date: Oct. 30, 2008

(54) Reaction Vessel

(75) Inventor: Ben Cobb, North Wraxall Wiltshire (GB)

Correspondence Address: WOLF GREENFIELD & SACKS, P.C., 600 ATLANTIC AVENUE, BOSTON, MA, 02210-2206, US

(73) Assignee: EVOGEN LTD., SURREY (GB)

(21) Appl. No.: 11/661,618

(22) PCT Filed: Aug. 30, 2005
(86) PCT No.: PCT/GB2005/050138

§ 371 Date: Jan. 11, 2008

(30) Foreign Application Priority Data
Aug. 31, 2004 (GB) 0419294.4

(51) Int. Cl. C12P 19/34; B01L 3/00; C12M 1/00; B29C 45/00

(52) U.S. Cl. 435/91.2; 422/102; 264/299; 435/283.1

(57) ABSTRACT

A reaction vessel for use in thermal cycling reactions is disclosed. The vessel has a width greater than its depth, to give the vessel a flattened profile. The side walls of the vessel extending across the width of the vessel are generally flat, and have a lesser thickness than the side walls extending across the depth of the vessel, This conformation allows rapid heat transfer into the vessel during thermal cycling, while the flattened profile allows optical detection techniques to be used on the contents of the vessel. The tubes may be provided singly or in an array or multiwell formal. Also described is a carousel for holding the tubes for use in a thermal cycler.

### FIELD OF THE INVENTION

The present invention relates to a reaction vessel, and to a method of manufacturing a reaction vessel. In particular, but not exclusively, the reaction vessel is intended for use in thermal cycling applications.

### BACKGROUND OF THE INVENTION

Thermal cycling applications are an integral part of contemporary molecular biology. For example, the polymerase chain reaction (PCR), which is used to amplify nucleic acids, uses a series of DNA melting, annealing, and polymerisation steps at different temperatures to greatly amplify the amount of DNA in a sample. Conventional PCR reactions proceed in a closed vessel, with amplification being confirmed by extracting a sample from the finished reaction and analysing the product by gel electrophoresis.

This conventional analysis technique requires that the user wait until the cycling has finished before being able to confirm that amplification is taking place; this can lead to delays in obtaining experimental data, for example when a cycling reaction must be repeated due to failure of amplification. For this reason, alternative methods of analysing PCR and other amplification products have been developed which may be used to measure amplification at an earlier stage of the reaction. One such alternative technique involves the incorporation of fluorescently labelled nucleotides into the reaction; as the DNA is amplified, so the intensity of fluorescence will increase. Detecting this fluorescence during the reaction can give a real-time indication of the progress of amplification. Many other molecular biology techniques make use of optical measurements to determine the progress of a reaction; for example, optical absorbance of a particular wavelength.

Measurement of fluorescence or other optical properties during progress of a reaction presents particular problems for the design of instrumentation and consumables. Conventional PCR reaction vessels are in the form of individual vessels having uniformly tapered conical portions, or take a multi-well plate format. Such vessels can present a relatively large cross section to illuminating and emitted light, so reducing the intensity of light able to be received at a detector. Further, the conical portions of such vessels enclose a relatively high volume of reaction mix, which therefore has a high thermal lag, leading to longer cycle times. Reduction in the volume of reaction mix can reduce this difficulty, but will reduce the amount of fluorescence produced by the reaction, so requiring more sensitive detectors.

The effect of thermal lag is also exacerbated by the thickness of the reaction vessel walls. Thin walled vessels are available, having walls down to around 0.5 mm thick, but limits on injection moulding technology tend to prevent conventional reaction vessels being produced having substantially thinner walls.

Embodiments of the present invention are intended to provide a reaction vessel particularly suited for use in monitoring of reactions during thermal cycling. Certain embodiments of the invention are intended to provide a reaction vessel having thinner walls than conventional vessels.

### SUMMARY OF THE INVENTION

According to a first aspect of the present invention, there is provided a reaction vessel having a top end defining an opening;

a bottom closed end;

the top and bottom ends defining a longitudinal axis of the vessel;

the vessel having a width and a depth transverse to the longitudinal axis;

wherein the width of the vessel is greater than the depth.

This provides for a reaction vessel having a ‘flattened’ profile, such that light travelling through the depth of the vessel is less attenuated than light travelling through the width. The distance through the depth of the vessel is also less than the distance through a non-flattened vessel of similar size and equivalent volume, so improving optical detection characteristics. In addition, the flattened profile creates a greater surface area for a given volume than an equivalent non-flattened reaction vessel. This exposes a greater surface area to heating and cooling when used in a thermal cycler, thereby assisting heat transfer to the reaction mix. This can reduce the length of time needed for each cycle.

Preferably the vessel has substantially flat walls extending along the width of the vessel. That is, the wider walls of the vessel are generally planar. The vessel also comprises narrower walls extending along the narrower depth of the vessel; these narrower walls may nonetheless have some degree of width, or the wider walls may meet to effectively form an edge. The flat walls of the vessel provide for improved optical properties, in that a larger viewing window is available, of a consistent curvature and depth, for transmission of light through the sample.

The vessel is preferably tapered along the longitudinal axis, narrowing from the top toward the bottom. This arrangement allows relatively small reaction mix volumes to extend some way up the vessel, so retaining the benefits of a high surface area for a given volume. Preferably the taper is found only in the width dimension of the vessel, and not in the depth dimension. This ensures that the depth dimension of the vessel is consistent along the longitudinal axis, so allowing consistent optical measurements to be taken at any point along this axis.

Preferably the vessel is constructed to funnel light toward the bottom of the vessel. That is, light entering the vessel at an angle other than along one of the axes will be guided by internal reflection toward the bottom of the vessel. This allows for even relatively low intensity light to be detected, and also permits detection of light without the need to accurately align the source and detector. It has been found that using a tapered shape for the vessel obtains acceptable results for light funneling.

Preferably the walls of the vessel extending along the width of the vessel have lesser thickness than the walls extending along the depth of the vessel. This reduced thickness improves heat transfer through these walls, so reducing the necessary cycle time in a thermal cycler. Such a construction is also robust, since the thicker depth axis walls provide strength and support to the thinner walls. Furthermore, this construction reduces the problems inherent in injection moulding; very thin walled tubes cannot conventionally be made by injection moulding, since sufficient space must be left between the mould walls to allow flow of the polymer used for manufacture. The present construction allows polymer to be injected along the thicker space of the thicker walls, and still flow to create the thinner walls of the vessel. In addition, injection moulding can require venting of gases; this venting can leave distortions in the injected polymer, reducing the effectiveness of optical measurements. The present construction allows venting to preferentially take place along the thicker walls, so leaving the thinner walls more suited to optical measurements.

Preferably the ratio of the thickness of the walls of the vessel extending along the depth of the vessel to the thickness of the walls extending along the width of the vessel is greater than 2:1, and more preferably around 3:1, and most preferably 8:3. In preferred embodiments of the invention, the walls extending along the depth of the vessel are less than or equal to 0.8 mm thick, while the walls extending along the width of the vessel are less than or equal to 0.3 mm thick.

The vessel is preferably formed from polypropylene, but other polymers may be used where suitable.

Preferably the vessel has a usable volume of 20 to 100 microlitres.

Preferably the vessel includes a flared upper section. The upper section may widen to a substantially greater depth than the remainder of the vessel, while the width may be substantially equal to the width of the remainder of the vessel. The flared upper section of the vessel allows access for standard pipette tips to add or remove reaction mix from the vessel.

The vessel may further comprise a cap for selectively closing the opening at the top end. The cap may be attached to the remainder of the vessel; for example, by means of a living hinge, or may be separate.

According to a further aspect of the present invention, there is provided a reaction vessel array comprising a plurality of reaction vessels, each vessel having a top end defining an opening; a bottom closed end; the top and bottom ends defining a longitudinal axis of the vessel; the vessel having a width and a depth transverse to the longitudinal axis; wherein the width of the vessel is greater than the depth.

The array may be in the form of a multi-well plate, a strip of vessels, or a ring or other closed loop of vessels. Individual vessels from the array are preferably joined to form a unitary array. A plurality of caps may also be provided, suitable for closing at least a plurality of vessels within the array. The caps may be joined to form a plate, strip, or ring.

According to a further aspect of the present invention, there is provided a method of producing a reaction vessel, the method comprising providing an injection mould shaped to form a vessel having a top end defining an opening; a bottom closed end; the top and bottom ends defining a longitudinal axis of the vessel; the vessel having a width and a depth transverse to the longitudinal axis; wherein the width of the vessel is greater than the depth; and

injecting a polymer into the mould, and allowing the polymer to cure.

Preferably the mould is shaped to form a vessel having walls extending along the width of the vessel of lesser thickness than walls extending along the depth of the vessel.

A further aspect of the present invention provides a method of conducting a thermal cycling reaction, the method comprising: placing a suitable reaction mix in a reaction vessel, the reaction vessel having a top end defining an opening; a bottom closed end; the top and bottom ends defining a longitudinal axis of the vessel; the vessel having a width and a depth transverse to the longitudinal axis; wherein the width of the vessel is greater than the depth; and

placing the reaction vessel in a thermal cycler; and

operating the thermal cycler.

The skilled person will be aware of suitable reaction mixes which may be used in a thermal cycling reaction; for example, standard PCR protocols and the like. Conventional thermal cyclers may also be used.

A still further aspect of the present invention provides a carousel for use in a thermal cycler, the carousel comprising a body having a plurality of through bores each shaped to accept a reaction vessel having a top end defining an opening; a bottom closed end; the top and bottom ends defining a longitudinal axis of the vessel; the vessel having a width and a depth transverse to the longitudinal axis; wherein the width of the vessel is greater than the depth;

the bores being arranged circumferentially around the body.

The bores are open, and allow air flow within the cycler to access the reaction vessels to ensure thermal transfer.

The carousel may be designed to be fitted to a conventional thermal cycler; the skilled person will appreciate that different thermal cyclers make use of different forms of carousel, and that modifications to the form of the carousel may be necessary to allow a carousel to be fitted to a particular cycler. However, such modifications are within the capabilities of the skilled person.

Preferably the bores of the carousel are arranged to accept reaction vessels such that the longitudinal axes of the vessels are not parallel with one another; that is, the reaction vessels are angled relative to the main axis of the carousel. This arrangement allows the vessels to act somewhat as vanes, in order to direct air flow within the thermal cycler; this has the advantage of improving air mixing, so resulting in more uniform thermal transfer. Even in embodiments in which the bores are not angled, the flattened shapes of the reaction vessels used in the carousel may still, to some extent, act as vanes.

### BRIEF SUMMARY OF THE DRAWINGS

These and other aspects of the present invention will now be described by way of example only and without limitation, with reference to the accompanying drawings, in which

FIGS. 1 and 2 are front and side external views of a reaction vessel in accordance with an embodiment of the present invention;

FIGS. 3 and 4 are sectional views of the reaction vessel of FIGS. 1 and 2 along lines A-A and X-X respectively; and

FIGS. 5, 6, and 7 are top, side sectional, and bottom views respectively of a carousel for use with reaction vessels of FIGS. 1 to 4.

### DETAILED DESCRIPTION OF THE DRAWINGS

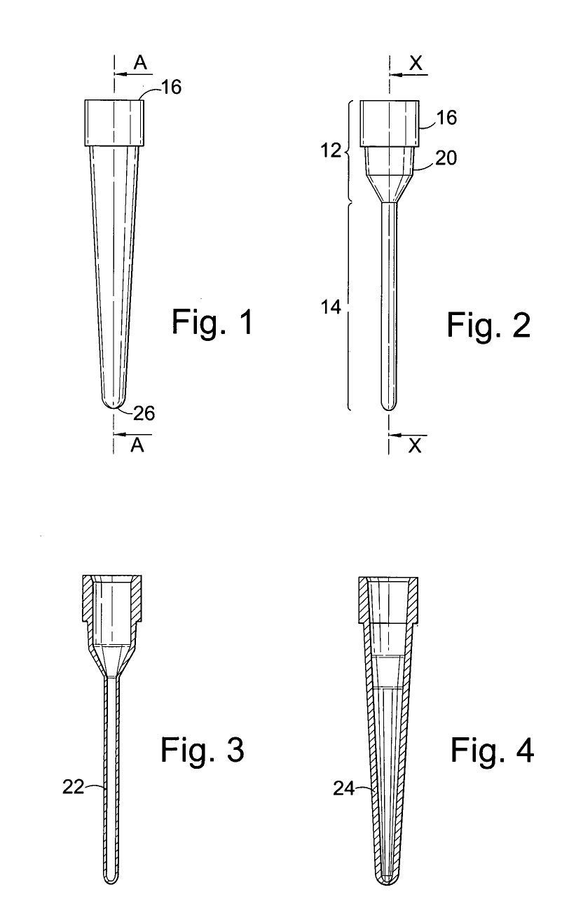

Referring to FIGS. 1 to 4, these show a reaction vessel 10 in accordance with an embodiment of the present invention. The vessel 10 includes an upper section 12 and a lower section 14. The upper section 12 includes an opening 16 allowing access to the interior of the vessel, while the lower section 14 terminates in a closed end 26 of the vessel. The axis of the vessel extending from the opening 16 to the closed end 26 defines a longitudinal axis; perpendicular to this axis are the width and depth of the vessel. As used herein, the width of the vessel refers to the axis perpendicular to the longitudinal axis along which the lower section 14 has its greater extent. The depth of the vessel refers to the axis perpendicular to the longitudinal axis along which the lower section 14 has its lesser extent. The width of the vessel is illustrated in FIGS. 1 and 3, while the depth is illustrated in FIGS. 2 and 4.

The upper section 12 includes a generally cylindrical portion 18 and a flared portion 20.

The upper section 12 joins the lower section 14 at the lower end of the flared portion 20. The lower section has a substantially unvarying depth (as can be seen in FIG. 2) along its length, and a greater width which narrows from the top of the lower section to the bottom (as can be seen in FIG. 1).

The cross sectional views of FIGS. 3 and 4 illustrate the varying wall thicknesses of the vessel. The thickness of the walls 22 extending across the width of the vessel (see FIG. 3) is much less than that of the walls 24 extending across the depth of the vessel (see FIG. 4). In this particular embodiment, the walls 22 are 0.3 mm thick, while the walls 24 are 0.8 mm thick. The flared upper section 12 has still thicker walls at its uppermost portion. The walls 22 can be seen to be substantially flat, while the walls 24 have a slight taper.

The vessel 10 may be used as follows. The flared upper section 12 is wide enough to permit access by standard pipette tips, which allows loading of the lower section 14 with reaction mix. Once the lower section is loaded, the constant depth of this section is reflected in a constant depth of reaction mix. In this embodiment, the maximum volume of the lower section is 100 microlitres, although the tapering nature of the width of this section allows volumes of between 20 and 100 microlitres to be loaded while retaining a substantial height of mix along the longitudinal axis of the vessel, even at low volumes.

The loaded vessel may then be placed in a thermal cycler. As thermal cycling occurs, the thin wall section 22 allows rapid heat transfer from the cycler to the reaction mix, while the high surface area to volume ratio of the reaction mix in the lower section also promotes rapid heat cycling of the reaction mix. The overall time needed for each cycle is therefore reduced compared with a similar reaction mix volume in a conventional reaction vessel.

The reaction vessel is also particularly suited to optical detection of reaction progress. The large, flat walls 22 provide large viewing windows which allow illumination of the reaction mix and detection of any emitted light. As is discussed below, the manufacturing process also helps to reduce optical imperfections in these walls 22. In addition, the tapering lower section of the vessel promotes internal reflection of light within the vessel, serving to funnel light along the vessel towards the closed end 26. This increases the intensity of light at this end of the vessel, thereby improving detection by detectors located toward this end. The internal reflection also allows for reduced accuracy in alignment of source and detector, since incident light at angles other than perpendicular will still be reflected and funneled toward the end of the vessel.

The vessel is manufactured by injection moulding of polypropylene. The injection is carried out along the thicker wall portions 24, with the thinner wall portions 22 being formed by flow of injected material from these thicker portions. This process allows for thinner wall sections to be created than would otherwise be possible. Further, venting of gases from the injection process is directed along the thicker wall portions, and kept away from the thinner wall portions. This ensures that any optical imperfections introduced by venting are confined to the thicker wall portions, leaving the thinner portions suitable for optical monitoring of a reaction. The thicker wall sections also serve to provide strength and robustness to the reaction vessel.

Although FIGS. 1 to 4 show a single reaction vessel, it will be apparent that multiple vessels may be formed in a single piece. For example, vessels may be formed in strips or rings, or in multiwell plate format. Vessels may also be formed with integral lids, for example, lids attached by living hinges to the upper portion of the vessel, or lids may be formed separately, for example, in a separate strip form to correspond with strips of reaction vessels.

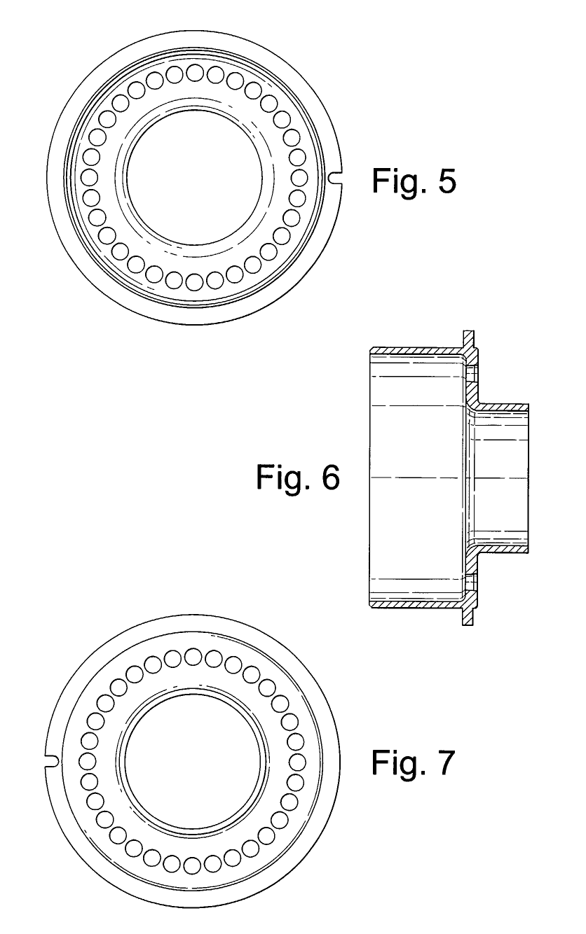

A carousel for supporting such vessels in a thermal cycler is shown in FIGS. 5 to 7. The carousel is a generally cylindrical block suitable for mounting in a particular cycler, with a number of through bores located radially around the block. In certain embodiments, the bores may be angled toward the central axis of the block, such that vessels mounted in the carousel are held at an angle to this axis. In use, the carousel may be mounted in a thermal cycler, and the carousel rotated selectively in order to locate a desired vessel adjacent a light source and detector. This provides for rapid detection of multiple reactions in a single process. The angling of the bores ensures that the flattened lower sections of the vessels are held to act as vanes for directing air flow within the cycler. This air flow can improve mixing of warm and cool air within a cycler which uses air heating, for example the LightCycler manufactured by Roche, Inc, thereby reducing thermal lag time during heating and cooling.

It will be apparent that the vessel of the present invention allows for improved thermal transfer during thermal cycling reactions, while the form of the vessel is also well suited to optical detection of reactions. The skilled person will appreciate that the embodiments described herein are illustrative only, and that various modifications and variations may be made to the vessel shown herein without departing from the scope of the invention.

1. A reaction vessel having a top end defining an opening;
a bottom closed end;
the top and bottom ends defining a longitudinal axis of the vessel;
the vessel having a width and a depth transverse to the longitudinal axis; wherein the width of the vessel is greater than the depth.

2. A vessel according to claim 1, wherein the vessel has substantially flat walls extending along the width of the vessel.

3. A vessel according to claim 1, wherein the vessel is tapered along the longitudinal axis.

4. A vessel according to claim 3, wherein the taper is found only in the width dimension of the vessel, and not in the depth dimension.

5. A vessel according to claim 1, wherein the vessel is constructed to funnel light toward the bottom of the vessel.

6. A vessel according to claim 1, wherein the walls of the vessel extending along the width of the vessel have lesser thickness than the walls extending along the depth of the vessel.

7. A vessel according to claim 6, wherein the ratio of the thickness of the walls of the vessel extending along the depth of the vessel to the thickness of the walls extending along the width of the vessel is greater than 2:1.

8. A vessel according to claim 7, wherein the ratio is around 3:1.

9. A vessel according to claim 1, wherein the walls extending along the depth of the vessel are less than or equal to 0.8 min thick, and the walls extending along the width of the vessel are less than or equal to 0.3 min thick.

10. A vessel according to claim 1 having a usable volume of 20 to 100 microlitres.

11. A vessel according to claim 1, wherein the vessel includes a flared ripper section.

12. A reaction vessel array comprising a plurality of reaction vessels, each vessel having a top end defining an opening; a bottom closed end; the top and bottom ends defining a longitudinal axis of the vessel; the vessel having a width and a depth transverse to the longitudinal axis; wherein the width of the vessel is greater than the depth.

13. A method of producing a reaction vessel, the method comprising providing an injection mold shaped to form a vessel having a top end defining an opening; a bottom closed end; the top and bottom ends defining a longitudinal axis of the vessel; the vessel having a width and a depth transverse to the longitudinal axis; wherein the width of the vessel is greater than the depth; and
injecting a polymer into the mold, and allowing the polymer to cure.

14. A method of conducting a thermal cycling reaction, the method comprising: placing a suitable reaction mix in a reaction vessel, the reaction vessel having a top end defining an opening; a bottom closed end; the top and bottom ends defining a longitudinal axis of the vessel; the vessel having a width and a depth transverse to the longitudinal axis; wherein the width of the vessel is greater than the depth; and
placing the reaction vessel in a thermal cycler; and
operating the thermal cycler.

15. A carousel for use in a thermal cycler, the carousel comprising a body having a plurality of through bores each shaped to accept a reaction vessel having a top end defining an opening; a bottom closed end; the top and bottom ends defining a longitudinal axis of the vessel; the vessel having a width and a depth transverse to the longitudinal axis; wherein the width of the vessel is greater than the depth;
the bores being arranged circumferentially around the body.

16. A carousel according to claim 15, wherein the bores of the carousel are arranged to accept reaction vessels such that the longitudinal axes of the vessels are not parallel with one another.
